(19) United States
(12) Patent Application Publication
(10) Pub. No.: US 2023/0390222 A1
(43) Pub. Date: Dec. 7, 2023

(54) Amyloid-Binding Peptoids with Broad-Spectrum Antiviral, Antibacterial, and Antifungal Activity

(71) Applicants: The Board of Trustees of the Leland Stanford Junior University, Stanford, CA (US); Sens Foundation, Inc., Mountain View, CA (US); The Regents of the University of California, Oakland, CA (US)

(72) Inventors: Annelise E. Barron, Woodside, CA (US); Michael J. Rae, Oakland, CA (US); Jevgenij Raskatov, Santa Cruz, CA (US); John A. Fortkort, Austin, TX (US)

(73) Assignees: The Board of Trustees of the Leland Stanford Junior University, Stanford, CA (US); Sens Foundation, Inc., Mountain View, CA (US); The Regents of the University of California, Oakland, CA (US)

(21) Appl. No.: 18/263,862

(22) PCT Filed: Feb. 1, 2022
(86) PCT No.: PCT/US2022/070465

§ 371 Date: Aug. 1, 2023

Related U.S. Application Data
(60) Provisional application No. 63/144,365, filed on Feb. 1, 2021.

(51) Int. Cl. A61K 31/16; A61K 47/12

(52) U.S. Cl. CPC A61K 31/16; A61K 47/12

(57) ABSTRACT

A method is provided for treating a subject suffering from a chronic infection that involves the brain. The chronic infection may range from those causing only mild cognitive impairment, to those present in individuals who have been diagnosed with Alzheimer's Disease. The method includes forming a composition containing (a) an anti-infective peptoid, and (b) a blood-brain barrier (BBB) manipulator that enhances the ability of the anti-infective peptoid to cross the BBB; and administering a therapeutic amount of the composition to a subject in need thereof.

### CROSS-REFERENCE TO RELATED APPLICATIONS

The current application claims priority to U.S. Provisional Patent Application No. 63/144,365, filed Feb. 1, 2021 entitled “Amyloid-Binding Peptoids with Broad-Spectrum Antiviral, Antibacterial, and Antifungal Activity” to Barron et al.; the disclosure of which is hereby incorporated by reference in its entirety.

### FIELD OF THE DISCLOSURE

The present disclosure relates generally to pharmaceutical compositions, and more particularly to compositions containing peptoid mimics of LL-37 which behave as broad-spectrum anti-infective compositions that also inhibit A3 amyloid formation.

### BACKGROUND OF THE DISCLOSURE

Poly-N-substituted glycines, also known as peptoids, are a class of peptidomimetics whose side chains are appended to the nitrogen atom of the peptide backbone, rather than to the α-carbons (as is the case in amino acids). Peptoids lack the amide hydrogen which is responsible for many of the secondary structure elements in peptides and proteins. As is the case with D-Peptides and p peptides, peptoids are resistant to proteolysis, and thus are advantageous in therapeutic applications where proteolysis is a major issue. Since the secondary structure in peptoids does not involve hydrogen bonding, it is not typically denatured by solvent, temperature, or chemical denaturants. Moreover, since the amino portion of the amino acid can utilize any amine, thousands of commercially available amines may be utilized to generate families of compounds with extensive chemical diversity at each position. One exemplary class of peptoids is described, for example, in U.S. Pat. No. 8,673,842 (Barron et al.; the disclosure of which is incorporated by reference herein in its entirety).

### SUMMARY OF THE DISCLOSURE

This summary is meant to provide some examples and is not intended to be limiting of the scope of the invention in any way. For example, any feature included in an example of this summary is not required by the claims, unless the claims explicitly recite the features. Various features and steps as described elsewhere in this disclosure may be included in the examples summarized here, and the features and steps described here and elsewhere can be combined in a variety of ways.

In one embodiment, a method is provided for treating a subject suffering from Alzheimer's disease, including forming a composition containing (a) an N-substituted glycine oligomer (peptoid), and (b) a blood-brain barrier (BBB) manipulator which enhances the ability of the peptoid to cross the BBB; and administering a therapeutic amount of the composition to a subject in need thereof.

In a further embodiment, a composition includes an N-substituted glycine oligomer (peptoid) and a blood-brain barrier (BBB) manipulator which enhances the ability of the peptoid to cross the BBB.

In another embodiment, the BBB manipulator is docosahexaenoic acid (DHA).

In a still further embodiment, the BBB manipulator further includes nanocarriers.

In still another embodiment, the nanocarriers are lipid nanocarriers.

In a yet further embodiment, the lipid nanocarriers comprises an amount of DHA within the range of 5-15%.

In yet another embodiment, the lipid nanocarriers have a maximum dimension within the range of 90 to 140 nm.

In a further embodiment again, the nanocarriers are selected from the group: polymeric nanoparticles, solid lipid nanoparticles, liposomes, micelles, dendrimers, nanogels, nanoemulsions and nanosuspensions.

In another embodiment again, the peptoid is H-(NLys-Nspe-Nspe)₄-NH₂.

In a further additional embodiment the peptoid includes a poly-N-substituted glycine compound of a formula

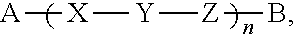

where A is a terminal N-alkyl substituted glycine residue, n is an integer, B is selected from the group consisting of NH2, one and two N-substituted glycine residues, and where said one and two N-substituted glycine residues have N-substituents which are independently selected from natural α-amino acid side chain moieties, isomers and carbon homologs thereof, and X, Y and Z are independently selected from the group consisting of N-substituted glycine residues, wherein said N-substituents are independently selected from the group consisting of natural α-amino acid side chain moieties, isomers and carbon homologs thereof, and proline residues.

In another additional embodiment, said alkyl substituent is selected from about C₄ to about C₂₀ linear, branched and cyclic alkyl moieties.

In a still yet further embodiment, n has a value within the range of 1-3.

In still yet another embodiment, at least one of said X, Y and Z residues is N_{Lys} and at least one said N-substituent is chiral.

In a still further embodiment again, at least one of Y and Z are proline residues.

In still another embodiment again, Y and Z are proline residues.

In a still further additional embodiment, A is a terminal N-alkyl substituted glycine residue, where said alkyl substituent selected from the group consisting of C₆ to about C₁₈ linear alkyl moieties, wherein B is NH₂, and wherein n is 1 or 2.

In still another additional embodiment, A is a terminal N-alkyl substituted glycine residue, said alkyl substituent selected from about C₆ to about C₁₈ linear alkyl moieties; where B is an N_{Lys} residue; and wherein n is 1.

In a yet further embodiment again, the compound is a hexamer.

In yet another embodiment again, the compound is a dodecamer.

In a yet further additional embodiment, at least one of A, B, X, Y and Z contains a halogen-bearing moiety.

In yet another additional embodiment, said halogen-bearing moiety contains a halogen-substituted aryl moiety.

In a further additional embodiment again, said halogen-bearing moiety contains a chloro-substituted aryl moiety.

In another additional embodiment again, said halogen-bearing moiety contains a bromo-substituted aryl moiety.

In a still yet further embodiment again, said halogen-bearing moiety contains an iodo-substituted aryl moiety.

In still yet another embodiment again, wherein each mer in the hexamer contains a halogen-substituted aryl moiety.

In a still yet further additional embodiment, some of the mers in the hexamer contain a halogen-substituted aryl moiety, and where some of the mers in the hexamer contain a halogen-free aryl moiety.

In still yet another additional embodiment, exactly one of the mers in the hexamer contains a halogen-substituted aryl moiety.

In a yet further additional embodiment again, each mer in the hexamer contains a halogen-substituted aryl moiety.

In yet another additional embodiment again, some of the mers in the hexamer contain a halogen-substituted aryl moiety, and wherein some of the mers in the hexamer contain a halogen-free aryl moiety.

In a still yet further additional embodiment again, only the first and last mers in the hexamer contain a halogen-substituted aryl moiety.

In still yet another additional embodiment again, at least two of A, B, X, Y and Z contain a halogen-bearing moiety.

In another further embodiment, all of A, B, X, Y and Z contain a halogen-bearing moiety.

In still another further embodiment, said pharmaceutical composition is a pharmaceutically acceptable salt of the poly-N-substituted glycine compound.

In yet another further embodiment, the poly-N-substituted glycine is H-(NLys-Nspe-Nspe)₄-NH₂.

In another further embodiment again, the poly-N-substituted glycine is Cy5.5-Ahx-(NLys-Nspe-Nspe)₄-NH₂.

Another further additional embodiment, the poly-N-substituted glycine is H-(NLys-Nspe-Nspe(p-Br))₂-NH₂.

In yet another further additional embodiment, the poly-N-substituted glycine is H-Ntridec-NLys-Nspe-Nspe-NLys-NH₂.

Yet again, in another additional embodiment, the poly-N-substituted glycine is H-(NLys-Nspe-Nspe)₃-NLys-Nspe-NH₂.

In still yet another further additional embodiment again, the poly-N-substituted glycine is H-(NLys-Nspe-Nspe)₂-NH₂.

In yet another further additional embodiment again, the poly-N-substituted glycine is H-Ndec-(NLys-Nspe-Ns7e)₂-NH₂.

Again, in yet another still further additional embodiment, the poly-N-substituted glycine is H-Ndec-(NLys-Nspe-Nspe(p-Br))₂—NH₂.

In yet another further additional embodiment again, the poly-N-substituted glycine is H-Ntridec-(NLys-Nspe-Nspe(p-Br))₂—NH₂.

Yet again, in a further additional embodiment, the peptoid is

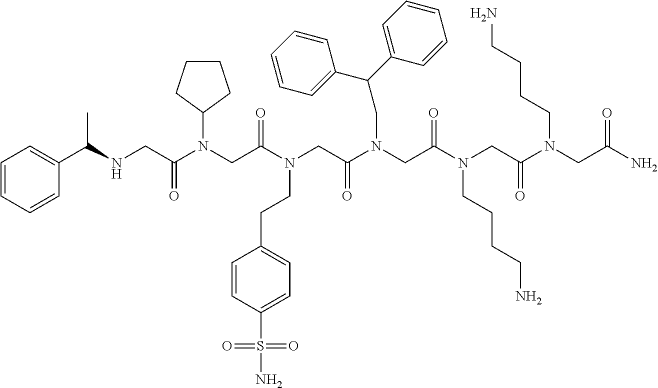

In yet a further additional embodiment again, the peptoid is

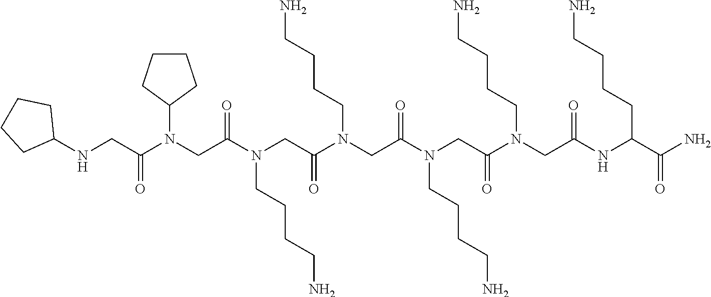 [SMILES: NCCCCC(NC(=O)CN(CCCCN)C(=O)CN(CCCCN)C(=O)CN(CCCCN)C(=O)CN(CCCCN)C(=O)CN(C(=O)CNC1CCCC1)C1CCCC1)C(N)=O]

### BRIEF DESCRIPTION OF THE DRAWINGS

FIGS. 1A-1C depict the results of Thioflavin T (Tht) fluorescence measurements of the process of Aβ amyloid formation following Aβ40 fibril formation. The results were obtained with Tht dye in the presence and absence of varying amounts of LL-37.

FIGS. 2A-2B is a series of TEM images of oligomers (left) and fibrils (right) of WT-Aβ40 (scale bar: 200 nm).

FIG. 3A is an SDS-PAGE followed by silver staining of fresh (0 h) and pre-incu-bated (7 d) Aβ42 and Aβ42-S26s. XL denotes PICUP crosslink.

FIG. 3B is a series of dot blots (6E10 antibody) of Aβ42 and Aβ42-S26s at varied time points post reconstitution. Sup: supernatant; pel: pellet.

FIG. 3C is a series of dot blots (OC) of fresh p3(17-40) (top) and Aβ40 (bottom).

FIG. 4 depicts familial Alzheimer's Disease (FAD) causing mutants that are located within the Aβ sequence.

FIG. 5 depicts a ThT fibril formation assay comparing fibril formation kinetics of Aβ40 wild-type and the Aβ40-E22Δ (Osaka) FAD mutant.

FIG. 6 is an illustration of the structure of Peptoid 1.

FIG. 7 is an illustration of the structure of a peptoid useful in the compositions and methods described herein.

FIG. 8 is an illustration of the structure of a peptoid useful in the compositions and methods described herein.

FIGS. 9-12 are graphs of intensity as a function of time for mixtures containing various concentrations of Peptoid 1 and Aβ40.

### DETAILED DESCRIPTION

Alzheimer's dementia (AD) is a societal health crisis. At present, 42 million elders suffer worldwide from AD, and the incidence of the disease is rising. Despite being under study for more than a century, the root causes of the onset of sporadic AD (which accounts for 95% of all cases) remain unclear. In order to prevent sporadic dementia, AD disease mechanisms and etiology will need to be better elucidated. Moreover, an approach to effectively treat sporadic AD, once it is diagnosed, has so far eluded researchers, which has led many pharmaceutical companies to abandon AD drug development. Indeed, despite the expenditure of a significant amount of research money and effort on AD worldwide, in the past 15 years, more than 420 clinical trials seeking FDA approval of AD therapies have failed. Meanwhile, older, FDA-approved drugs are effective at best only in staving off some of the symptoms of AD.

Amyloid R (A3) is a key protein involved in the pathogenesis of AD. Aβ is produced through sequential cleavage of the precursor protein Amyloid Precursor Protein (APP) through β- and γ-secretase and yields a ˜36-43 amino acid (AA) long processing product. The 40 AA long isoform, Aβ40, is formed predominantly, but the less common 42 AA long isoform, Aβ42, is more toxic due to its higher aggregation propensity. Aβ oligomeric intermediates are believed to be particularly toxic.

Past therapeutic efforts have aimed at reducing Aβ levels in the brain, either by targeting Aβ directly, or by intervening with APP processing. Until recently, every treatment targeting Aβ failed in phase III clinical trials. In late 2019, the Aducanumab antibody that binds soluble Aβ oligomers and fibrils showed some limited benefit in a phase III clinical trial. Targeting soluble, toxic forms of Aβ remains a promising avenue for AD therapeutic development, but needs to be substantially improved to be clinically useful. Among the main drawbacks of Aβ-targeting antibodies are (a) the lack of understanding what structures they target and (b) the need for large amounts of antibodies to be infused to achieve a therapeutic dose in the brain. For example, Aducanumab needs to be infused at 4 g daily for a 150-pound patient to achieve its effects. Small molecules targeting toxic forms of Aβ directly would be much more attractive and viable from a clinical standpoint.

Recently, it has become clear that sporadic AD (not resulting from a unique familial predisposition to disease) is either caused by, or accompanied by the occurrence of, polymicrobial brain infections and dysfunction of the blood-brain barrier (BBB). Certain oral pathogens that are associated with dementia, including Porphyromonas gingivalis (PG) and Herpes Simplex Virus-1 (HSV-1), can be killed or inactivated by LL-37 peptide, which is produced by the human innate immune system (but only weakly). Moreover, high levels of LL-37 in the brain can cause damaging neuroinflammation, while systemically high levels of LL-37 are associated with autoimmune conditions including psoriasis and lupus erythematosus. Although Aβ:LL-37 interactions offer an interesting starting point in the development of new therapeutic approaches that block Aβ aggregation and toxicity, the relatively complex, poorly understood, pleiotropic immunomodulatory effects of LL-37, as well as its relatively high molecular weight (˜4500 g/mol, 37 amino acids) and extreme vulnerability to cleavage by proteases, are substantially disadvantageous features from the standpoint of using the peptide as an exogenous AD therapeutic.

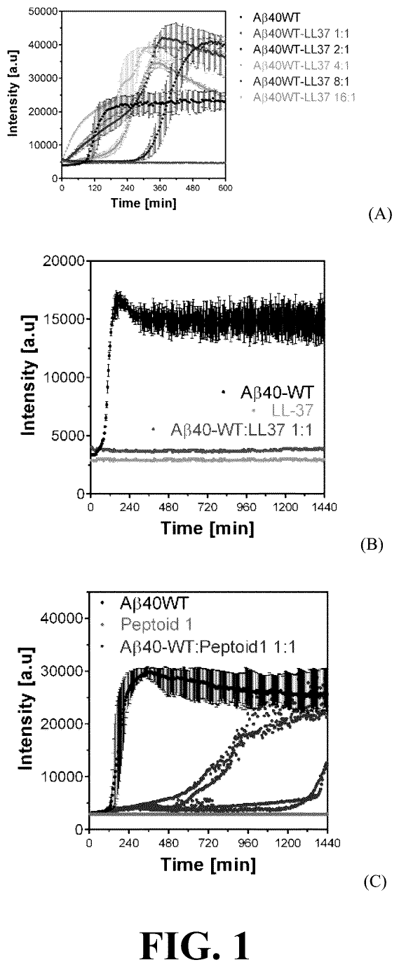

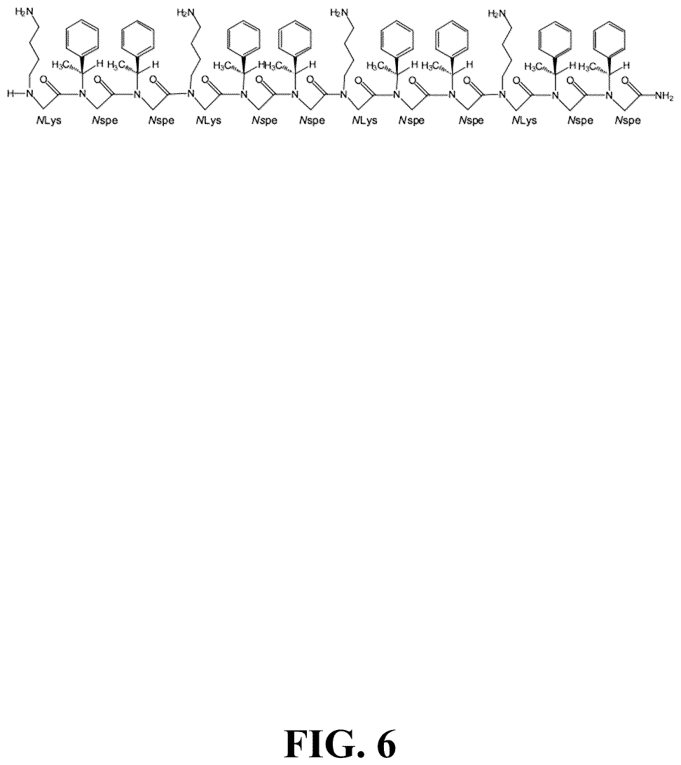

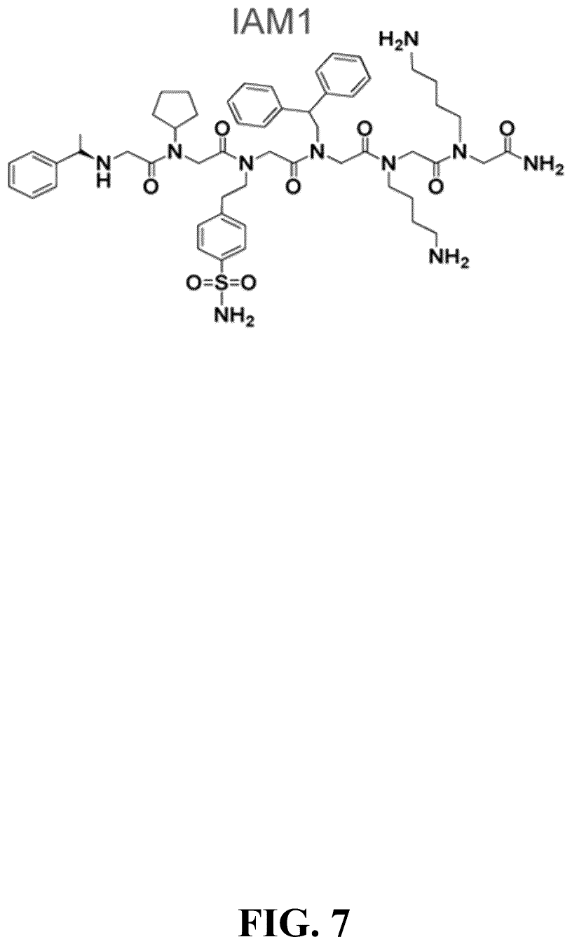

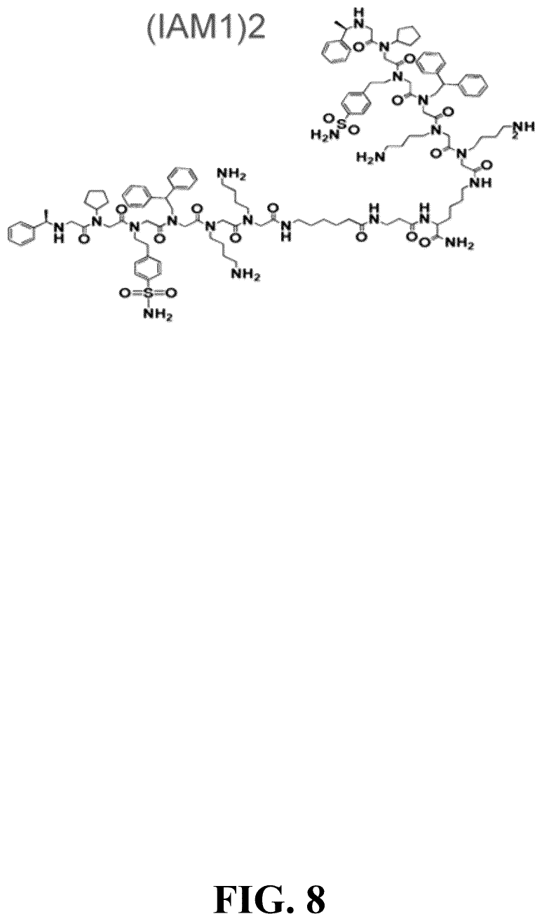

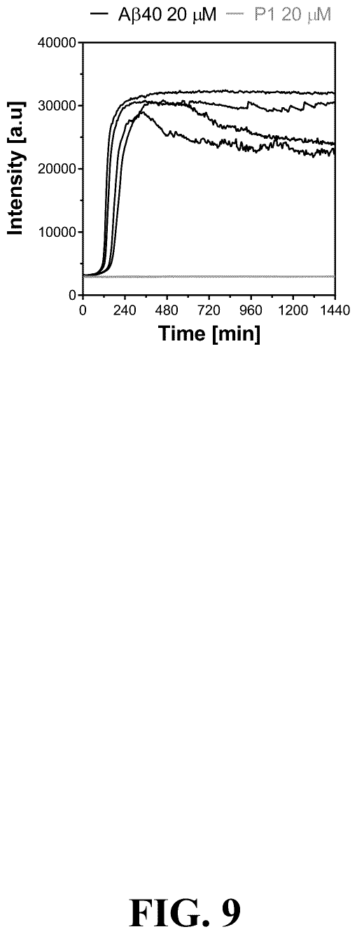

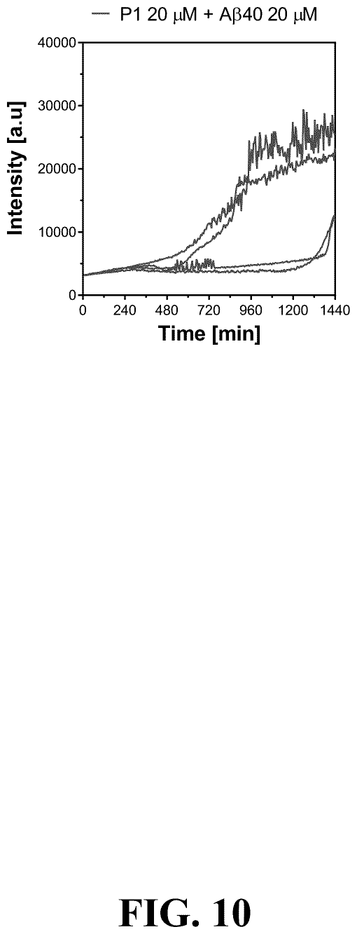

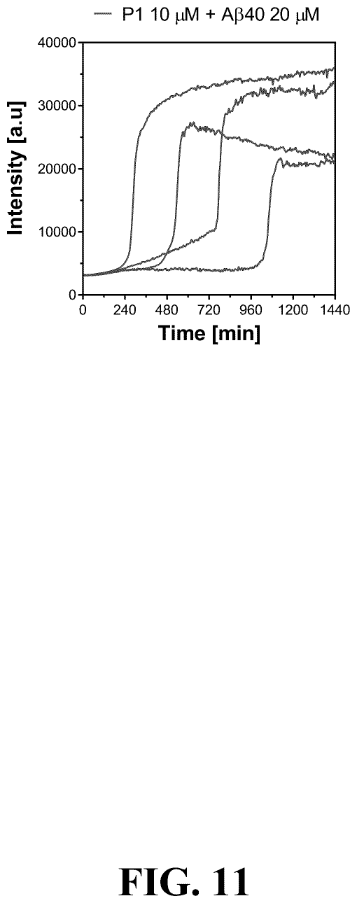

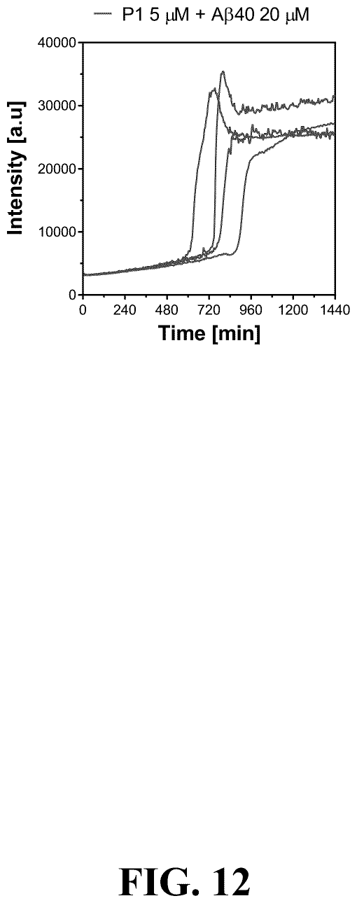

FIGS. 1A-1C depict exemplary results of Thioflavin T (Tht) fluorescence measurements of the process of Aβ amyloid formation following Aβ40 fibril formation. These results were obtained with Tht dye in the presence and absence of varying amounts of LL-37. As seen therein, 1:1 equimolar LL-37/Aβ40 mixtures are totally prevented from forming Aβ fibrils, while lower relative molar amounts of LL-37 slow the kinetics of fibril formation. It was also recently discovered that a peptoid mimic of LL-37 known as Peptoid 1 (see FIG. 6 for the chemical structure), shares the same anti-amyloid activity. This particular compound is of interest in that it has been found to exhibit potent (4 μg/mL) antimicrobial and antiviral activity against both P. gingivalis and HSV-1, two pathogens that are associated with dementia in humans. Peptoid 1 is also active against many other pathogens (e.g., P. aeruginosa, S. aureus, M tuberculosis, C. albicans, etc.), Thus, Peptoid 1 has the potential to be developed as an antimicrobial treatment that could also serve as an anti-amyloid treatment. As such, various embodiments utilize Peptoid 1 as an antimicrobial treatment. Additional embodiments provide Peptoid 1 orally, nasally, inhalationally, parentally, intravenously, intraperitoneally, subcutaneously, intramuscularly, intradermally, topically, rectally, intracerebrally, intraventricularly, intracerebroventricularly, intrathecally, intracisternally, intraspinally, perispinally, or combinations thereof. Due to the stability, or resistance to degradation, of peptoids, certain embodiments provide Peptoid 1 orally to allow absorption and systemic treatment (including into the brain) of an individual.

The results of preliminary in vitro studies show that the addition of LL-37 peptide in a 1:1 molar ratio to wildtype Aβ40 blocks fibril formation, whereas addition of sub-stoichiometric amounts slows it down in a dose-dependent manner. Final fluorescence values in ThT fibril formation assays that resulted from sub-stoichiometric LL-37 addition lead to a higher fluorescence output at endpoint. These results suggest that fibrils being formed under those conditions are of a different type. These results do not necessarily mean that there is more fibrillary material that has been formed when fluorescence output is higher. In particular, different fibrils bind ThT in different ways, and this may lead to differences in fluorescence output. This effect is important to understand in greater detail.

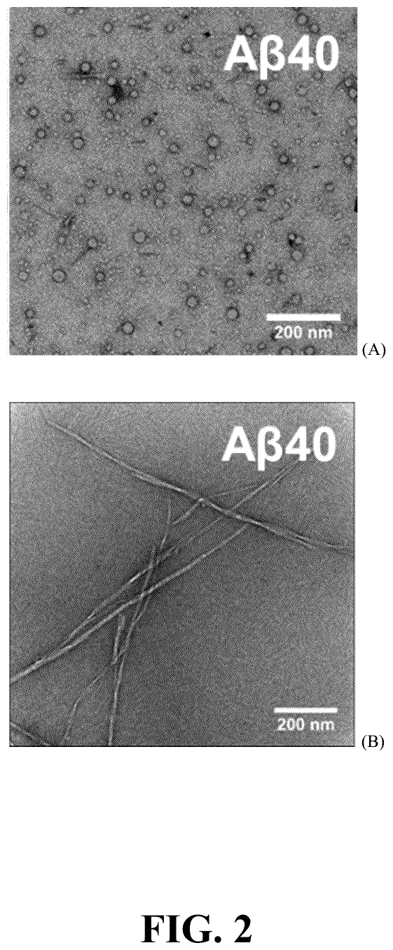

A key property of Aβ that is now believed to be at the core of its toxicity in AD is its ability to form soluble, toxic oligomers. Various embodiments provide assays that allow the measurement of oligomer formation and the elucidation of the impact of LL-37 addition on Aβ oligomerization. FIG. 2 illustrates TEM images of Aβ oligomers after a low temperature incubation protocol was utilized to stabilize the Aβ oligomers. Certain embodiments utilize a protocol such as this to determine the influence of LL-37, added in different relative amounts to A3, on oligomer formation. Further embodiments utilize quantitation scripts to compare the distributions thus obtained. Oligomer formation may also be monitored using the photochemically induced crosslinking of unmodified proteins (PICUP) assay. This latter approach allows for the analysis of smaller oligomeric aggregates using polyacrylamide gel electrophoresis, a method with high resolution of small analytes. The results obtained through this PICUP technique are complementary to those obtained with low temperature trapping followed by TEM analysis. Various embodiments use one or both techniques to obtain a detailed understanding of Aβ oligomerization in presence of different amounts of LL-37. These experiments may be performed either freshly upon reconstitution, or after 7 days of incubation to determine the effect of aging on those preparations.

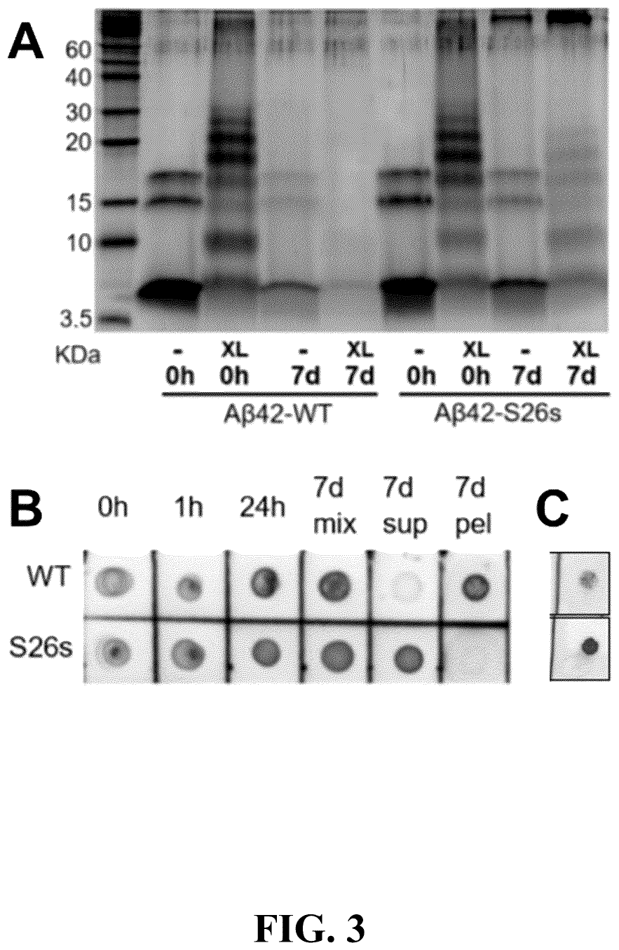

FIGS. 3A-3C illustrate exemplary results of stabilization and aggregation assays, such as those described herein. Specifically, FIG. 3A illustrates that, under certain conditions, soluble aggregation intermediates can be stabilized for multiple days, while FIG. 3B illustrates dot blot results using an OC antibody that recognizes Aβ fibrillary species. Through the combination of ThT fibril formation assays, TEM morphological analyses, PICUP electrophoretic assays and antibody-based dot blot experiments, an in-depth understanding may be obtained of how the biophysical process of Aβ oligomerization and aggregation proceeds in the presence of different relative (molar) amounts of LL-37.

While FIGS. 2-3 illustrate exemplary data using Aβ40. Various embodiments of these assays can be performed on the Aβ42 isoform. The Aβ42 isoform is more aggregation-prone, more toxic and also believed to be more important in AD. Once the assays have been optimized with the Aβ40 isoform, the corresponding experiments may be performed with Aβ42, which is more AD-relevant. The resulting optimized assays of these embodiments can then be utilized to study the effects of the LL-37-mimetic peptoids on Aβ oligomer and fibril formation, and to compare those effects with those observed with natural LL-37.

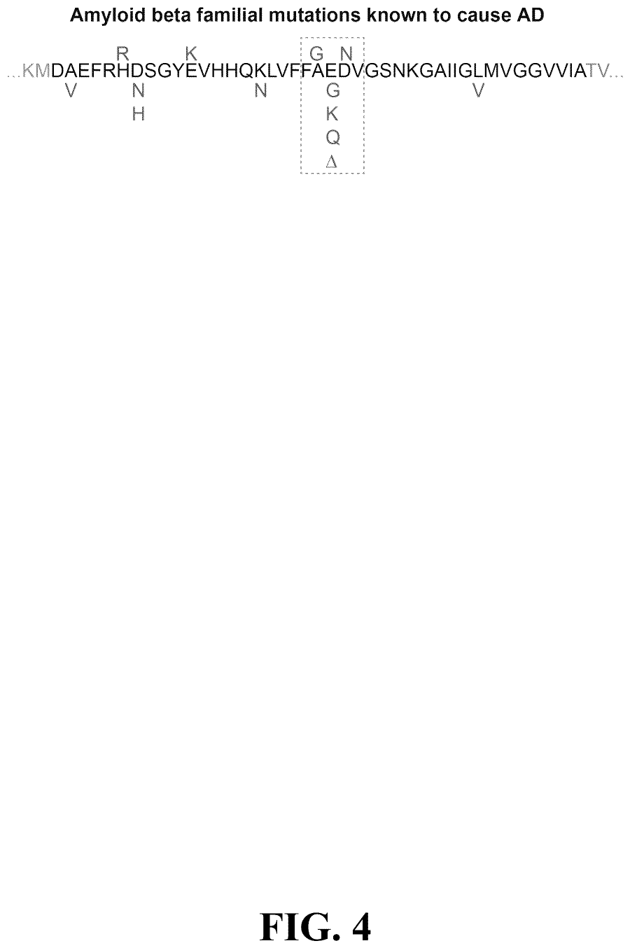

Familial Alzheimer's Disease (FAD) is known to cause amino acid mutations that are located within the Aβ peptidic framework. These mutations are changes at the single amino acid residue level which can lead to a much more aggressive disease phenotype that is typically guaranteed and sets on at an earlier age than if one does not carry the mutations. Some exemplary mutations are summarized in FIG. 4. It is notable that many of those mutations alter the net anionic charge of Aβ from −3 to either −2 (e.g., H6R, D7N, D8H, K16N, E22G, E22Q, E22Δ, D23N) or to −1 (e.g., E11K, E22K), thus bringing it closer to neutral. Without wishing to be bound by theory, it is believed that Coulombic attraction plays an important role in the interactions between Aβ and LL-37, and it is further believed that these charge-altering Aβ mutations will lead to lower attraction between the mutant forms of Aβ with LL-37, and to disruption in the ability of LL-37 to neutralize Aβ toxicity. This may be one significant reason (in addition to their accelerated aggregation kinetics) that these FAD-causing mutations are more toxic. Certain embodiments are capable of testing such a hypothesis via one or more assay described herein Aβ wildtype peptides also for the Aβ40-E22Δ, the FAD-causing Aβ variant known as the Osaka mutation.

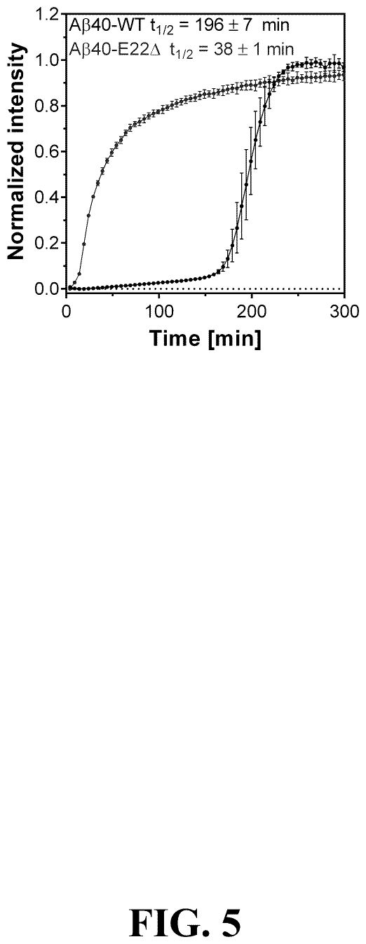

FIG. 5 illustrates exemplary data of preliminary experiments in which Aβ40-E22Δ was synthesized and subjected to standard conditions utilized in ThT fibril formation assays. FIG. 5 illustrates that the deletion of the E22 residue leads to a pronounced acceleration of fibril formation, which dropped from 196±7 min with Aβ40-WT to 38±1 min with Aβ40-E22Δ. The Aβ40-E22Δ mutant is much more amyloidogenic than Aβ40-WT, which may complicate efforts to block its amyloidogenicity.

The exemplary studies illustrated in the figures, where Aβ amyloid formation was monitored as a function of time, may be repeated and subsequently expanded upon to elucidate and compare how the LL-37 mimic Peptoid 1 and other variant peptoid mimics of LL-37 effectively prevent and/or slow the formation and accumulation of amyloid based on the Alzheimer's-associated Aβ peptide. Turning to FIG. 1C, exemplary results representing the initial testing of Peptoid 1 mimicry of LL-37's ability to prevent Aβ amyloid formation. In this exemplary embodiment, the assay was initially performed at 20 μM peptide/peptoid concentrations. Further embodiments vary the concentrations of the peptide and/or peptoid to identify dose effects and/or effective dose sizes of the peptoid of certain embodiments (e.g., Peptoid 1). Quantitative mathematical analyses of the effects that various molar ratios of LL-37 vs. Peptoid 1 have on the kinetics of Aβ amyloid formation may be performed. It is to be noted that the effects on Aβ amyloid formation of Peptoid 1 are more “stochastic” and less precisely repeatable than what was seen with LL-37. A greater understanding of this result, possibly coupled with suitable modifications of the molecular design of Peptoid 1, may yield highly repeatable results, such as those seen for LL-37.

In the human body, there are constant opposing processes of amyloid formation vs. amyloid clearance. If a certain, well-tolerated dose of Peptoid 1 can slow the rate of amyloid formation, it may allow the body's macrophages and microglia to effectively clear amyloid from the body's tissues via the process of macroautophagy, restoring them to better health. If (as has been found in recent research) amyloid in the brain is in fact encapsulating, sequestering, and inactivating certain pathogens such as HSV-1 virus and Porphyromonas gingivalis bacteria, it may still be necessary to slow the development of fresh amyloid in order to clear this pathogen-enrobing amyloid from the body. It is to be noted that Peptoid 1 is an active, broad-spectrum antimicrobial compound which is effective in killing P. gingivalis bacteria at 4 μg/mL, and in totally inactivating HSV-1 virus at 4 μg/mL as well. Those preliminary studies may be extended to P. gingivalis biofilms as well as planktonic (free-swimming) P. gingivalis bacteria.

Certain embodiments utilize rodent models of Alzheimer's disease to test whether Peptoid 1 treatment is well tolerated and/or is efficacious. Some embodiments use at least two different rodent models of Alzheimer's for this purpose. The 5×FAD mouse model, which overexpresses human Aβ amyloid peptide into its brain, may be utilized to determine whether Peptoid 1 is able to prevent amyloid accumulation in the 5×FAD mouse brain, starting with young (2-month-old) mice (since the goal would be to prevent amyloid formation). By the age of 4-5 months, 5×FAD mice develop a substantial amyloid burden). A wildtype mouse model of P. gingivalis oral and brain infection (created by applying the human-relevant P. gingivalis W83 strain of bacteria to wildtype mice, orally, MWF each week for 22 weeks) may also be utilized. If left untreated, wildtype mice infected with P. gingivalis will become chronically infected with P. gingivalis in both their mouths and brain tissue. This chronic infection results in six observable differences from healthy wildtype mice: neuroinflammation, neurodegeneration, microgliosis, astrogliosis and the formation of intra- and extracellular Aβ amyloid plaque and phosphorylated Tau (pTau) neurofibrillary tangles, all of which are part of the pathology of AD (see e.g., doi.org/10.1371/journal.pone.0204941; the disclosure of which is incorporated by reference herein in its entirety).

This animal model may be utilized to test whether Peptoid 1 is able to treat the oral infection of these wild-type mice with the P. gingivalis W83 strain of pathogenic bacteria, thus preventing amyloid and pTau accumulation in the brain.

In preferred embodiments of the compositions and methodologies disclosed herein, a composition is provided which contains a peptoid. Further embodiments include a blood-brain barrier (BBB) manipulator to enhance the ability of the peptoid to cross the BBB. However, certain dementias disrupt the BBB and/or peptoids of some embodiments can cross the BBB (freely or with minimal resistance), thus making a BBB manipulator superfluous in various embodiments. A therapeutic amount of this composition may be administered to an AD patient or other subject in need thereof.

The compounds disclosed herein can exist as therapeutically acceptable salts. The term “therapeutically acceptable salt,” as used herein, represents salts or zwitterionic forms of the compounds disclosed herein which are therapeutically acceptable as defined herein. The salts can be prepared during the final isolation and purification of the compounds or separately by reacting the appropriate compound with a suitable acid or base. Therapeutically acceptable salts include acid and basic addition salts. For a more complete discussion of the preparation and selection of salts, refer to “Handbook of Pharmaceutical Salts, Properties, and Use,” Stah and Wermuth, Ed., (Wiley-VCH and VHCA, Zurich, 2002) and Berge et al, J. Pharm. Sci. 1977, 66, 1-19.

In some embodiments, active ingredients are administered in a therapeutically effective amount as part of a course of treatment. As used in this context, to “treat” means to ameliorate at least one symptom of a disorder to be treated or to provide a beneficial physiological effect. For example, one such amelioration of a symptom could be reduction of risk of spontaneous preterm labor, spontaneous abortion, recurrent preterm birth, or recurrent pregnancy.

A therapeutically effective amount can be an amount sufficient to prevent reduce, ameliorate or eliminate the symptoms of gestational complications susceptible to such treatment.

Dosage, toxicity and therapeutic efficacy of the compounds can be determined, e.g., by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds that exhibit high therapeutic indices are preferred. While compounds that exhibit toxic side effects may be used, care should be taken to design a delivery system that targets such compounds to the site of affected tissue in order to minimize potential damage to other tissue and organs and, thereby, reduce side effects.

Data obtained from cell culture assays or animal studies can be used in formulating a range of dosage for use in humans. If the pharmaceutical is provided systemically, the dosage of such compounds lies preferably within a range of circulating concentrations that include the ED50 with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration or within the local environment to be treated in a range that includes the ED50 as determined in cell culture or animal models. Such information can be used to more accurately determine useful doses in humans. Levels in plasma may be measured, for example, by mass spectrometry.

An “effective amount” is an amount sufficient to effect beneficial or desired results. For example, a therapeutic amount is one that achieves the desired therapeutic effect. This amount can be the same or different from a prophylactically effective amount, which is an amount necessary to prevent onset of disease or disease symptoms. An effective amount can be administered in one or more administrations, applications or dosages. A therapeutically effective amount of a composition depends on the composition selected. The compositions can be administered from one or more times per day to one or more times per week; including once every other day, as determined to be beneficial. The skilled artisan will appreciate that certain factors may influence the dosage and timing required to effectively treat a subject, including but not limited to the severity of the disease or disorder, previous treatments, the general health and/or age of the subject, and other diseases present. Moreover, treatment of a subject with a therapeutically effective amount of the compositions described herein can include a single treatment or a series of treatments. For example, several divided doses may be administered daily, one dose, or cyclic administration of the compounds to achieve the desired therapeutic result.

Preservatives and other additives, like antimicrobial, antioxidant, chelating agents, and inert gases, can also be present. (See generally, Remington: The Science and Practice of Pharmacy, 21st Edition; Lippincott Williams & Wilkins: Philadelphia, P A, 2005.)

The BBB manipulator is preferably a surfactant, and is most preferably docosahexaenoic acid (DHA). The BBB manipulator may comprise nanocarriers, which may be selected from the group consisting of polymeric nanoparticles, solid lipid nanoparticles, liposomes, micelles, dendrimers, nanogels, nanoemulsions and nanosuspensions. The nanocarriers are preferably lipid nanocarriers. The lipid nanocarriers preferably comprises an amount of DHA within the range of 5-15%, and preferably have a maximum dimension within the range of 90 to 140 nm.

Various peptoids and oligomers of N-substituted glycines may be utilized in accordance with the teachings herein to make the pharmaceutical compositions disclosed herein. In addition to the peptoids set forth in TABLE 1 below, these include the peptoids described in U.S. Pat. No. 8,445,632 (Barron et al.), which is incorporated herein by reference in its entirety; the peptoids disclosed in U.S. Pat. No. 9,938,321 (Kirshenbaum et al.), U.S. Pat. No. 9,315,548 (Kirshenbaum et al.) and U.S. Pat. No. 8,828,413 (Kirshenbaum et al.), all of which are incorporated by reference herein in their entireties; and the peptoids described in Nam, H. Y., Choi, J., Kumar, S. D., Nielsen, J. E., Kyeong, M., Wang, S., Kang, D., Lee, Y., Lee, J., Yoon, M.-H., Hong, S., Lund, R., Jenssen, H., Shin, S. Y., Seo, J., 2020. Helicity Modulation Improves the Selectivity of Antimicrobial Peptoids. ACS Infectious Diseases 6, 2732-2744. doi:10.1021/acsinfecdis.0c00356, which is incorporated by reference herein in its entirety.

TABLE 1
Peptoid Samples

| compound | Reference | project |
|---|---|---|
| H-(NLys-Nspe-Nspe)₄-NH₂ | TM1 | Peptoid 1 |
| Cy5.5-Ahx-(NLys-Nspe-Nspe)₄-NH₂ | TM1-Cy5.5 | |
| H-(NLys-Nspe-Nspe(p-Br))₂-NH₂ | TM2 | |
| H-NLys-Nspe-Nspe-NLys-Nspe-Nspe(p-Br)-NH₂ | TM3 | |
| H-(NLys-Nspe(p-Br)-Nspe(p-Br))₂-NH₂ | TM4 | |
| H-Ntridec-NLys-Nspe-Nspe-NLys-NH₂ | TM5 | |
| H-(NLys-Nspe-Nspe)₃-NLys-Nspe-NH₂ | TM6 | |
| H-(NLys-Nspe-Nspe)₂-NH₂ | TM7 | |
| H-Ndec-(NLys-Nspe-Nspe)₂-NH₂ | TM8 | |
| H-Ndec-(NLys-Nspe-Nspe(p-Br))₂-NH₂ | TM9 | |
| H-Ntridec-(NLys-Nspe-Nspe(p-Br))₂-NH₂ | TM10 | |
| H-(NLys-Nspe-Nspe)₄-NLys-NH₂ | TM11 | Peptoid1 + NLys |
| H-(NLys-Nspe-Nspe)₃-NLys-Nspe-NLys-NH₂ | TM16 | |
| H-(NLys-Nspe-Nspe)₂-NLys-NH₂ | TM17 | |
| H-NLys-Nspe-Nspe-NLys-Nspe-Nspe(p-Br)-NLys-NH₂ | TM13 | |
| H-(NLys-Nspe-Nspe(p-Br))₂-NLys-NH₂ | TM12 | |
| H-(NLys-Nspe(p-Br)-Nspe(p-Br))₂-NLys-NH₂ | TM14 | |
| H-Ntridec-NLys-Nspe-Nspe-NLys-NLys-NH₂ | TM15 | |
| H-Ndec-(NLys-Nspe-Nspe)₂-NLys-NH₂ | TM18 | |
| H-Ndec-(NLys-Nspe-Nspe(p-Br))₂-NLys-NH₂ | TM19 | |
| H-Ntridec-(NLys-Nspe-Nspe(p-Br))₂-NLys-NH₂ | TM20 | |
| H-(NLys-NLeu-NLeu)₃-NLys-NLeu-NH₂ | TM21 | controls |
| H-(NLys-Nssb-Nssb)₄-NH₂ | TM22 | |

One class of the foregoing peptoids which are useful in the compositions and methodologies described herein are the poly-N-substituted glycine compound of a formula

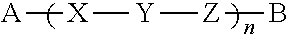

wherein
A is a terminal N-alkyl substituted glycine residue,
n is an integer,
B is selected from the group consisting of NH₂, one and two N-substituted glycine residues, and wherein said one and two N-substituted glycine residues have N-substituents which are independently selected from natural α-amino acid side chain moieties, isomers and carbon homologs thereof, and
X, Y and Z are independently selected from the group consisting of N-substituted glycine residues, wherein said N-substituents are independently selected from the group consisting of natural α-amino acid side chain moieties, isomers and carbon homologs thereof, and proline residues.

Various other N-substituted glycine oligomers or peptoid” may be utilized in the compositions and methodologies disclosed herein. These include the poly-N-substituted glycine H-Ntridec-(NLys-Nspe-Nspe(p-Br))₂-NH₂, as well as the peptoids

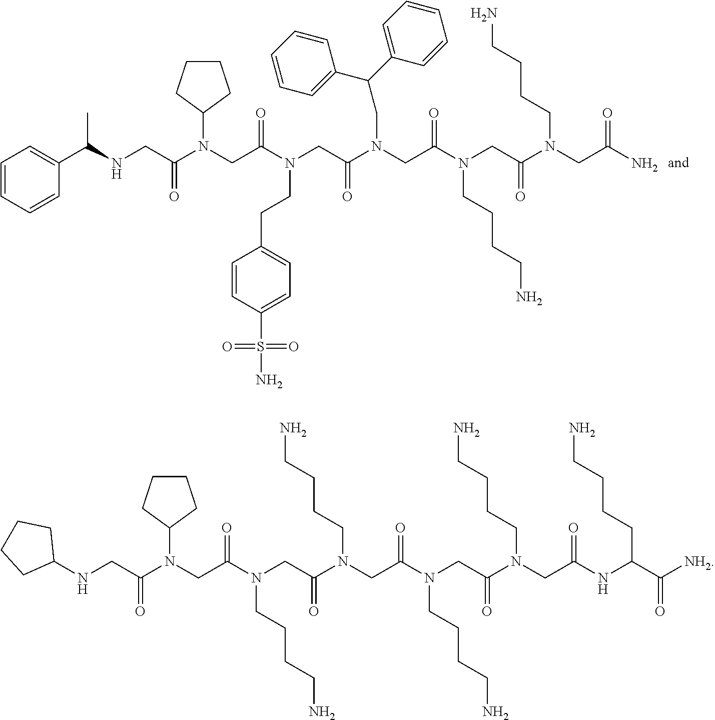

Various halogenated peptoids and halogenated oligomers of N-substituted glycines may also be utilized in accordance with the teachings herein to make antiviral pharmaceutical compositions and treatments. These include, without limitation, various halogenated analogs of the foregoing peptoids and oligomers of N-substituted glycines. These halogenated compositions may be halogenated in various ways. For example, these compounds may include any number of halogen substitutions with the same or different halogens. In particular, these compounds may include one or more fluoro-, chloro-, bromo- or iodo-substitutions, and may include substitution with two or more distinct halogens. However, the use of one or two bromo- or chloro-substitutions is preferred in many applications. Moreover, while the peptoids described herein may be halogenated at various locations, para halogenation on the peptoids containing aryl rings is especially preferred in many applications, although ortho- and meta-substitution, or even perhalogentation, may be useful in some applications.

The compositions described herein may also be alkylated, and preferably have terminal alkylation. Here, alkylation (and especially terminal alkylation) with a C10 or C13 tail is especially preferred. It has been found that such terminal alkylation can dramatically enhance the antibacterial activity of a peptoid, and in some cases, may cause a peptoid which otherwise has low antibacterial activity to have significant antibacterial activity.

The compositions described herein may include various other materials beside peptoids and BBB manipulators. Thus, for example, these compositions may include various pharmaceutically acceptable excipients, adjuvants, carriers, buffers or stabilizers.

### DOCTRINE OF EQUIVALENTS

Having described several embodiments, it will be recognized by those skilled in the art that various modifications, alternative constructions, and equivalents may be used without departing from the spirit of the invention. Additionally, a number of well-known processes and elements have not been described in order to avoid unnecessarily obscuring the present invention. Accordingly, the above description should not be taken as limiting the scope of the invention.

Those skilled in the art will appreciate that the foregoing examples and descriptions of various preferred embodiments of the present invention are merely illustrative of the invention as a whole, and that variations in the components or steps of the present invention may be made within the spirit and scope of the invention. Accordingly, the present invention is not limited to the specific embodiments described herein, but, rather, is defined by the scope of the appended claims.

1. A method for treating a subject, comprising:
administering to a subject suffering from Alzheimer's disease or mild cognitive impairment a therapeutic amount of a composition containing
(a) an N-substituted glycine oligomer (peptoid), and
(b) a blood-brain barrier (BBB) manipulator which enhances the ability of the peptoid to cross the BBB.

2. The method of claim 1, wherein the BBB manipulator comprises docosahexaenoic acid (DHA).

3. The method of claim 7, wherein the BBB manipulator further comprises lipid nanocarriers.

4. (canceled)

5. The method of claim 3, wherein the lipid nanocarriers comprises an amount of DHA within the range of 5-15% by weight.

6. The method of claim 3, wherein the lipid nanocarriers have a maximum dimension within the range of 90 to 140 nm.

7. The method of claim 1, wherein the BBB manipulator comprises nanocarriers, and wherein the nanocarriers are selected from the group consisting of polymeric nanoparticles, lipid nanocarriers, solid lipid nanoparticles, liposomes, micelles, dendrimers, nanogels, nanoemulsions and nanosuspensions.

8. The method of claim 1, wherein the peptoid is H-(NLys-Nspe-Nspe)₄-NH₂.

9. The method of claim 1, wherein the peptoid comprises a poly-N-substituted glycine compound of a formula 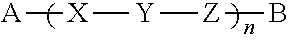
wherein A is a terminal N-alkyl substituted glycine residue, n is an integer, B is selected from the group consisting of NH₂, one and two N-substituted glycine residues, and wherein said one and two N-substituted glycine residues have N-substituents which are independently selected from natural α-amino acid side chain moieties, isomers and carbon homologs thereof, and X, Y and Z are independently selected from the group consisting of N-substituted glycine residues, wherein said N-substituents are independently selected from the group consisting of natural α-amino acid side chain moieties, isomers and carbon homologs thereof, and proline residues.

10. (canceled)

11. The method of claim 9, wherein n has a value within the range of 1-3.

12. The method of claim 9, wherein at least one of said X, Y and Z residues is N_{Lys} and at least one said N-substituent is chiral.

13-14. (canceled)

15. The method of claim 9, wherein A is a terminal N-alkyl substituted glycine residue, wherein said alkyl substituent selected from the group consisting of C₆ to about C₁₈ linear alkyl moieties, wherein B is NH₂, and wherein n is 1 or 2.

16. The method of claim 9, wherein A is a terminal N-alkyl substituted glycine residue, said alkyl substituent selected from about C₆ to about C₁₈ linear alkyl moieties; wherein B is an N_{Lys} residue; and wherein n is 1.

17-18. (canceled)

19. The method of claim 9, and wherein at least one of A, B, X, Y and Z contains a halogen-bearing moiety.

20. The method of claim 19, wherein said halogen-bearing moiety contains a halogen-substituted aryl moiety.

21. The method of claim 19, wherein said halogen-bearing moiety contains a chloro-substituted or bromo-substituted aryl moiety.

22-24. (canceled)

25. The method of claim 19, wherein at least one of the mers in the hexamer contain a halogen-substituted aryl moiety, and wherein at least one of the mers in the hexamer contain a halogen-free aryl moiety.

26-29. (canceled)

30. The method of claim 19, wherein at least two of A, B, X, Y and Z contain a halogen-bearing moiety.

31. (canceled)

32. The method of any of claim 9, wherein said pharmaceutical composition is a pharmaceutically acceptable salt of the poly-N-substituted glycine compound.

33. The method of claim 9, wherein the poly-N-substituted glycine is selected from the group consisting of H-(NLys-Nspe-Nspe)₄-NH₂, Cy5.5-Ahx-(NLys-Nspe-Nspe)4-NH₂, H-(NLys-Nspe-Nspe(p-Br))₂-NH₂, H-Ntridec-NLys-Nspe-Nspe-NLys-NH₂, H-(NLys-Nspe-Nspe)₃-NLys-Nspe-NH₂, H-(NLys-Nspe-Nspe)₂-NH₂, H-Ndec-(NLys-Nspe-Ns7e)₂-NH₂, H-Ndec-(NLys-Nspe-Nspe(p-Br))₂-NH₂, H-Ntridec-(NLys-Nspe-Nspe(p-Br))₂-NH₂,

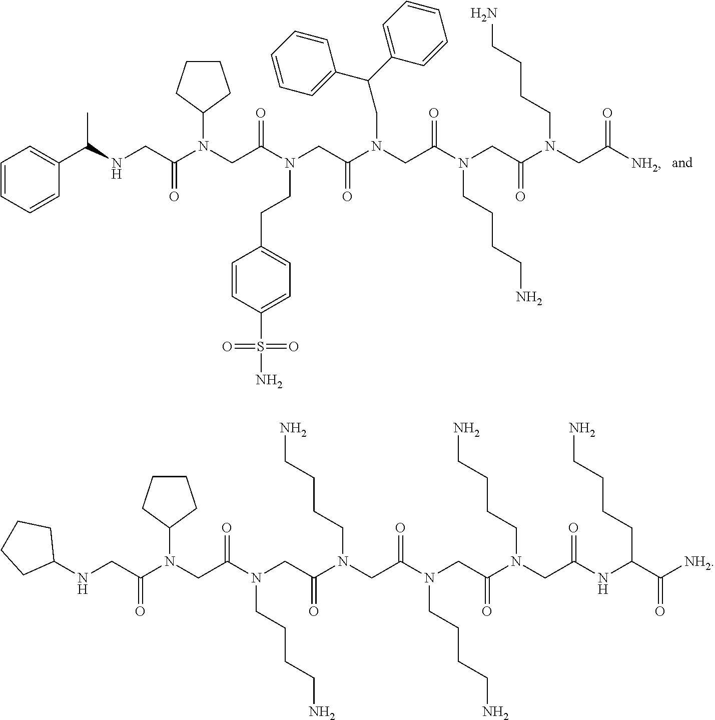

34-43. (canceled)

44. A composition, comprising:
an N-substituted glycine oligomer (peptoid); and
a blood-brain barrier (BBB) manipulator which enhances the ability of the peptoid to cross the BBB.

45-86. (canceled)
